(19) United States
(12) Patent Application Publication
(10) Pub. No.: US 2016/0331303 A1
(43) Pub. Date: Nov. 17, 2016

(54) METHODS AND SYSTEMS FOR SNORE DETECTION AND CORRECTION

(71) Applicant: ENTANTI LIMITED, San Diego, CA (US)

(72) Inventors: Haoye Shen, San Diego, CA (US); Bahman Khatam, Escondido, CA (US); Zhan Li, San Diego, CA (US)

(73) Assignee: ENTANTI LIMITED, San Diego, CA (US)

(21) Appl. No.: 15/112,412

(22) PCT Filed: Jan. 22, 2015
(86) PCT No.: PCT/US2015/012494

§ 371 Date: Jul. 18, 2016

Related U.S. Application Data
(60) Provisional application No. 61/930,105, filed on Jan. 22, 2014.

(51) Int. Cl. A61B 5/00; A61B 5/1455

(52) U.S. Cl. CPC A61B 5/4806; A61B 5/746; A61B 5/7455; A61B 5/14551; A61B 5/681; A61B 5/0022; A61B 5/742

(57) ABSTRACT

A method of detecting snoring includes: at a first smart mobile device: receiving a first audio signal from a microphone of the first smart mobile device; determining that the first audio signal includes characteristics of snoring; and in response to a determination that the first audio signal includes characteristics of snoring: transmitting a signal to an external alert unit.

### CROSS-REFERENCE TO RELATED APPLICATIONS

This application claims the benefit of U.S. Provisional Application No. 61/930,105, filed Jan. 22, 2014, the content of which is incorporated by reference herein in its entirety.

### FIELD

This disclosure relates to smart mobile device and systems for snore detection and correction.

### BACKGROUND

Some traditional snore prevention devices use a mouthpiece or a denture device placed inside a user's mouth during sleep. The objective is to change throat muscle positions and thereby preventing snoring. These types of devices can be uncomfortable.

Other solutions, such as use of an electrical shock or a loud noise to awake the user, can be annoying and unpleasant. Loud noises can be particularly problematic when the user shares a bed or room with another.

Others, such as DE200810032410, employ radio communication between a detector and an alerting module. Using a dedicated sound detector and radio channel increases overall cost and can be unreliable. Such a design fails to record or track historical data for future analysis.

### SUMMARY

Disclosed herein are systems and methods that utilize a smart mobile device to detect a snore and send a signal to an alert unit to provide a stimulus to a user. The systems and methods described here can be relatively inexpensive (for example, utilizing a smart mobile device that is already owned by the user) and reliable.

A wearable alert unit alerts the user when the smart mobile device detects a snoring sound through the smart mobile device's built in microphone and/or receives a measured oxygen level from an external device. If the measured oxygen level is lower than a predetermined level, alerting commands are sent to the altering unit via standard Bluetooth or Bluetooth Low Energy channels or other wireless protocols and profiles. Upon receiving the alerting commands from the smart mobile device, the wearable alert unit generates a vibration stimulus to alert the user to change body position/pose and stop snoring. Both snoring sound and oxygen level are recorded in the smart mobile device during the night and can be transmitted over the wireless network that the smart mobile device supports including WiFi, 3G or 4G cellular communication to a remote cloud server for real time monitoring or future analysis.

Aside from other health related concerns and bedtime annoyance, snoring during sleep has impact on sleeping quality and over time, it can make people feel un-rested and tired during the day. The system herein disclosed and described provides a comfortable and easy to use solution for the users to utilize a lightweight wearable alert unit that could be attached to the user's body as a wristband, or a necklace, or an armband or a chest band. A smart mobile device such as a smart phone or a tablet computer with application software is used to detect the surrounding sound as well as to receive the measured blood oxygen level from the wearable alerting device and perform analysis to identify if the received information represents a snoring profile or if the blood oxygen level meets the predetermined level. Upon identification and qualification of a snore, the smart mobile device will send alerting commands to the alert unit via Bluetooth or Bluetooth Low Energy channels to alert the user. The level of the stimulus escalates over defined time intervals, until the user changes body position or otherwise stops snoring.

The microphone built in the smart mobile device is used to pick up snoring sound. The Wearable device may have a built-in oximeter to measure the blood oxygen level. In some embodiments, the oximeter is a pulse oximeter. The measured oxygen data is sent to the smart mobile device via wireless channels. The application software in the smart mobile device can support all major operating systems including, but not limited to, iOS, Android, and Windows. The alert unit may have built-in rechargeable battery to provide the power to the electronic circuit and the alerting transducer within the alert unit. The alerting transducer may generate vibration at various levels and frequencies as the stimulus to alert the user when he/she snores. The alert unit receives the alerting commands from the smart mobile device via a Bluetooth or Bluetooth Low Energy channels and protocols. The alerting stimulus strength starts at a low level and increases with defined time intervals if the user does not stop snoring. When smart mobile device and the application software detect that the user has stopped snoring, the alerting signal may stop and the alerting signal strength can be reset to the initial low level.

In a multiple user case (i.e., two users in the same room want to monitor and correct snoring) two smart mobile devices are placed near to each user. The devices communicate to each other via wireless communication to determine which user is snoring and then send the alerting commands to the appropriate wearable alerting device.

In one aspect, a method of detecting snoring includes: at a first smart mobile device: receiving a first audio signal from a microphone of the first smart mobile device; determining that the first audio signal includes characteristics of snoring; and in response to a determination that the first audio signal includes characteristics of snoring: transmitting a signal to an external alert unit.

In another aspect, determining that the first audio signal includes characteristics of snoring includes: receiving a communication from a second smart mobile device, wherein the communication comprises information about a second audio signal captured by the second smart mobile device; and determining whether at least one of the first audio signal and second audio signal is associated with a user of the first smart mobile device; in response to a determination that at least one of the first audio signal and second audio signal is not associated with a user of the first smart mobile device: foregoing transmission of the signal to the external alert unit; in response to a determination that at least one of the first audio signal and second audio signal is associated with a user of the first smart mobile device: transmitting the signal to the external alert unit. In a further aspect, determining whether at least one of the first audio signal and the second audio signal is associated with a user of the first smart mobile device comprises determining, from the information in the communication, at least one of a time of capture of the second audio signal at the second smart mobile device and an amplitude of the second audio signal.

In another aspect, the alert unit is a wearable unit. In another aspect, the alert unit is a vibratory unit.

In another aspect, the method includes receiving a measurement of an oxygen level of a user and wherein determining that the first audio signal includes characteristics of snoring further comprises determining that the oxygen level is lower than a predetermined threshold. In a further aspect, receiving a measurement of an oxygen level comprises receiving the measurement from the alert unit.

In another aspect, the first audio signal is recorded and the recorded audio signal is transmitted to an external server.

In another aspect, the method includes: receiving a third audio signal from the microphone of the first smart mobile device; determining that the third audio signal includes characteristics of snoring; and in response to a determination that the third audio signal includes characteristics of snoring: transmitting a second signal to an alert unit. In a further aspect, the second signal includes an instruction to increase an intensity of a stimulus produced by the alert unit. In a further aspect, the method includes determining whether a first snore associated with the first audio signal and a second snore associated with the third audio signal occurred within a predetermined period of time, and wherein the second signal includes an instruction to increase an intensity of a stimulus produced by the alert unit.

In another aspect, determining that the first audio signal includes characteristics of snoring comprises determining that at least one of a frequency, an amplitude, and a duration of the first audio signal meets a predetermined threshold.

In one aspect, a method of detecting snoring includes: at a first smart mobile device: receiving a first audio signal from a microphone of the first smart mobile device; receiving a communication from a second smart mobile device, wherein the communication comprises information about a second audio signal captured by the second smart mobile device; determining whether the first audio signal includes characteristics of snoring; determining whether at least one of the first audio signal and second audio signal is associated with a user of the first smart mobile device; in response to a determination that (a) at least one of the first audio signal and second audio signal is associated with a user of the first smart mobile device and (b) the first audio signal includes characteristics of snoring: providing an alert; and in response to a determination that (a) at least one of the first audio signal and second audio signal is associated with a user of the first smart mobile device or (b) the first audio signal does not include characteristics of snoring: foregoing providing an alert.

In another aspect, providing an alert comprises transmitting a signal to an external alert unit.

In another aspect, determining whether the second audio signal is associated with a user of the first smart mobile device comprises determining, from the information in the communication, at least one of a time of capture of the second audio signal at the second smart mobile device and an amplitude of a second audio signal.

In one aspect, a non-transitory computer-readable storage medium stores one or more programs, the one or more programs comprising instructions, which when executed by a first smart mobile device, cause the device to: determine that a first audio signal, received from a microphone of the first smart mobile device, includes characteristics of snoring; and in response to a determination that the first audio signal includes characteristics of snoring: transmit a signal to an external alert unit.

In one aspect, a non-transitory computer-readable storage medium stores one or more programs, the one or more programs comprising instructions, which when executed by a first smart mobile device, cause the device to: determine that a first audio signal, received from a microphone of the first smart mobile device, includes characteristics of snoring; determine whether at least one of the first audio signal and second audio signal, detected by a second smart mobile device, is associated with a user of the first smart mobile device; in response to a determination that (a) at least one of the first audio signal and second audio signal is associated with a user of the first smart mobile device and (b) the first audio signal includes characteristics of snoring: provide an alert; and in response to a determination that (a) at least one of the first audio signal and second audio signal is associated with a user of the first smart mobile device or (b) the first audio signal does not include characteristics of snoring: forego providing an alert.

### BRIEF DESCRIPTION OF THE DRAWINGS

FIG. 1 illustrates a system for snore detection and correction, in accordance with one aspect.

FIG. 2 illustrates a system for snore detection and correction, in accordance with one aspect.

FIG. 3 illustrates an exemplary smart mobile device for monitoring and analyzing audio signal and transmitting alert signals, in accordance with one aspect.

FIG. 4 illustrates an apparatus for holding a smart mobile device on a bed headboard so that the smart mobile device is close to the user, in accordance with one aspect.

FIG. 5 illustrates a wristband or an armband alert unit, in accordance with one aspect.

FIG. 6 illustrates a wristband alert unit, in accordance with one aspect.

FIG. 7 illustrates an alert unit integrated with a wrist-watch, in accordance with one aspect.

FIG. 8 illustrates a system block diagram of an alert unit, in accordance with one aspect.

FIG. 9 illustrates a software architecture for detection, analyzing and identifying snore sound, in accordance with one aspect.

FIG. 10 illustrates a software flowchart of architecture implemented in the smart mobile device, in accordance with one aspect.

FIG. 11 illustrates a cloud server configuration of connecting to a smart mobile device to store and share data for future analysis, in accordance with one aspect.

FIG. 12 illustrates a method of snore detection and correction, in accordance with one aspect.

FIG. 13 illustrates a method of snore detection and correction, in accordance with one aspect.

### DETAILED DESCRIPTION

In the following description of embodiments, reference is made to the accompanying drawings which form a part hereof, and in which it is shown by way of illustration specific embodiments in which the claimed subject matter may be practiced. It is to be understood that other embodiments may be utilized and structural changes may be made without departing from the scope of the claimed subject matter.

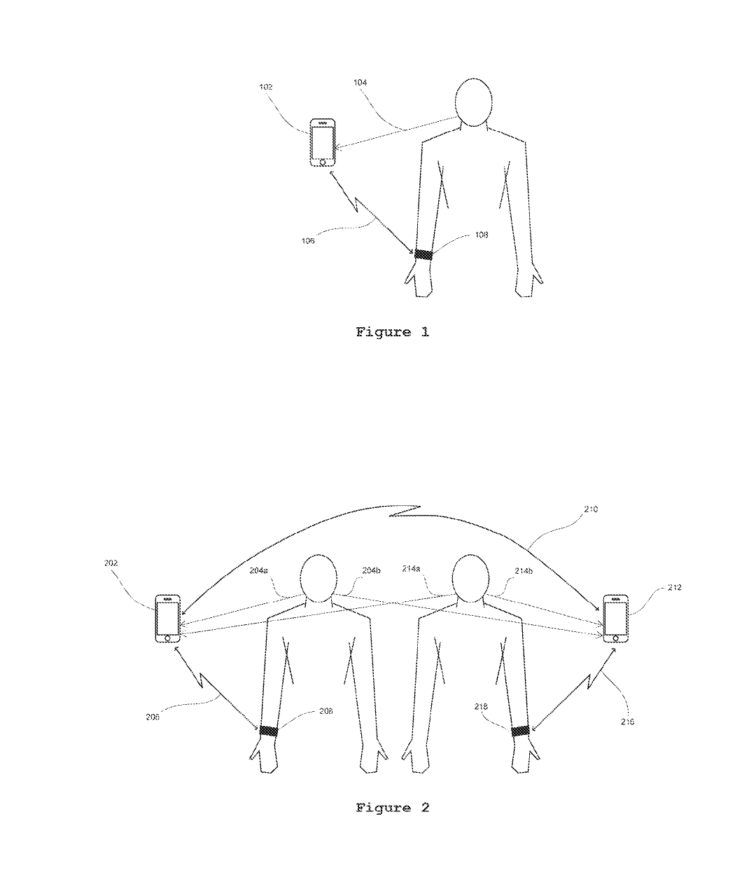

FIG. 1 illustrates a system 100 for snore detection and correction of one user, in accordance with one aspect. A smart mobile device 102 is positioned in proximity to a user. The device 102 receives an audio signal from a microphone (not shown) in the device. The audio signal represents a sound wave 104 emanating from the user.

Device 102 determines whether the audio signal includes characteristics of snoring (exemplary processes for determining whether an audio signal includes characteristics of snoring are described herein). If device 102 determines that the audio signal does include characteristics of snoring, then device 102 sends a signal 106 to an external alert unit 108. If device 102 determines that the audio signal does not include characteristics of snoring, then device 102 foregoes sending a signal to an external alert unit 108.

Because smart mobile devices are owned by most people, the system 100 will be low cost and easy to implement for most. In addition, by utilizing a smart mobile device, information on a patient's sleeping habits can be efficiently delivered to a storage system in the Cloud or to a health professional for further analysis.

FIG. 2 illustrates a system 200 for snore detection and correction of two users, in accordance with one aspect. A first smart mobile device 202 is positioned in proximity to a first user and a second smart mobile device 212 is positioned in proximity to a second user. Device 202 receives a first audio signal from a microphone (not shown) in device 202, where the first audio signal represents a first sound wave 204a emanating from the first user. Device 212 receives a second audio signal from a microphone (not shown) in device 212, where the second audio signal represents a second sound wave 204b emanating from the first user.

Device 202 receives a communication 210 from device 212, wherein the communication comprises information about the second audio signal 204b captured by the device 212. Device 202 determines whether the first audio signal includes characteristics of snoring. If device 202 determines that the audio signal does not include characteristics of snoring, then device 202 foregoes sending a signal to an external alert unit 208 associated with the first user. If device 202 determines that the audio signal does include characteristics of snoring, then device 202 determines whether at least one of the first audio signal and second audio signal is associated with a user of the first smart mobile device. If device 202 determines at least one of the first audio signal and second audio signal is associated with a user of the first smart mobile device, then device 202 sends a signal 206 to an external alert unit 208. In some embodiments, device 202 determines that both the first audio signal and second audio signal are associated with a user of the first smart mobile device, and then device 202 sends a signal 206 to an external alert unit 208

Device 212 also receives a communication from device 202, wherein the communication comprises information about the first audio signal 204a captured by the device 202. Device 212 follows an analogous process as device 202: determine if the second audio signal includes characteristics of snoring; if so, determine if the second audio signal is associated with the second user, etc.

System 200 may beneficially identify the correct source of the snore. This will improve the user experience by ensuring a snore-less user is not unnecessarily disturbed and the snoring user is alerted. In this way, co-sleepers can continue their normal sleeping arrangements while utilizing a snore detection and correction system.

In some embodiments, device 202 may use a time of capture of the audio signals to determine whether the audio signals are from the first user: a smart mobile device that is placed closer to a user may capture the snore first. Thus, by determining which capture time is earlier, device 202 can determine which user snored. In some embodiments, an amplitude of audio signals 204a and 204b may be compared to determine which is larger. A smart mobile device that is placed closer to a user will capture an audio signal with a larger amplitude. These mechanisms for determining whether the audio signal is associated with a first user are given by way of example, and should not be understood as limiting the scope of this disclosure.

FIG. 2 also illustrates sound waves 214a and 214b emanating from the second user and a signal 216 send from the second device 212 to a second external alert unit 218. This arrangement represents a scenario where a snore emanated from the second user and device 212 determined that the snore was associated with the second user, then sent a signal 216 to the second external alert unit 218.

Communication signal 210 may be direct (as illustrated in FIG. 2) or indirect (such as through a server). In some embodiments, devices 202 and 212 establish a wireless connection via a Bluetooth channel and set protocol at beginning of a communication session.

In some embodiments, if the system detects both users of the snore detection and correction system are snoring simultaneously, an alerting signal is sent to both alerting devices. In some embodiments where the system detects both users of the snore detection and correction system are snoring, the system may wait for the breathing rates of the two snorers to change so that the snore of each user can be individually identified (and then stored for tracking or analysis purposes, for example).

Although FIG. 2 illustrates two users of the snore detection and correction system, the system could be readily expanded to three or four users, as will be appreciated by one of skill in the art.

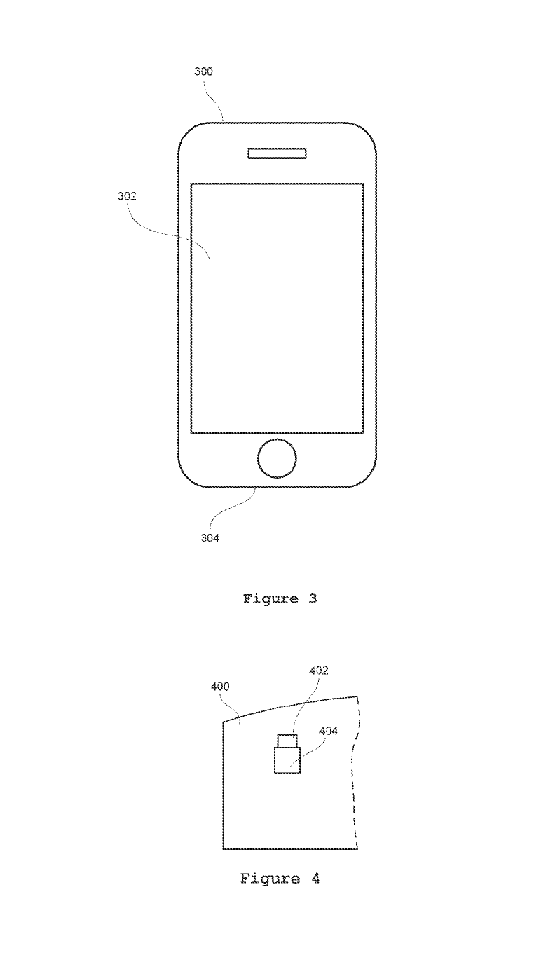

FIG. 3 illustrates an exemplary smart mobile device 300 for monitoring and analyzing audio signal and transmitting alert signals, in accordance with one aspect. Device 300 may be used as a sound sensor and analyzer unit in monitoring and analyzing snores. During operation, device 300 can be placed near to the user's head, such as on a bedside table or hung on the headboard with a holding device (FIG. 4).

Device 300 includes a display unit 302 to allow the user access the application software interface (not shown) on the display 302 to control the overall system as well as to review the collected historical data on the device or from a cloud server (see FIG. 11). The built-in microphone 304 may be used to capture the snoring sound signal and then the signal is processed by the smart mobile device processor with the application software. Once a snore sound signal is identified and qualified, the mobile device 300 transmits a signal to an alert unit, via a Bluetooth transceiver for example.

The application software within the mobile device can be used to define the alerting scheme. The scheme defines the alerting signal level, alerting signal duration, and altering signal pattern. The smart mobile device may record the snoring sound signal with a high compression algorithm to save memory space. The recorded sound signal can be used for future analysis and tracking for improvement.

In some embodiments, the alerting signal level starts at a lower value and escalates with a defined interval in case the user does not respond to the low alerting signal. An instruction may be sent in the signal to the alert unit to increase the stimulus. In some embodiments, the alert unit may determine when to increase the stimulus. When a user responds to the alerting signal and stops snoring, the smart mobile device will detect such status change and terminates the alerting command sent to the alert unit.

FIG. 4 illustrates an apparatus 400 for holding a smart mobile device 402 on a bed headboard so that the smart mobile device 402 is close to the user, in accordance with one aspect. In some embodiments, the apparatus 400 includes a pocket or holding pouch 404 into which the smart mobile device 402 is inserted.

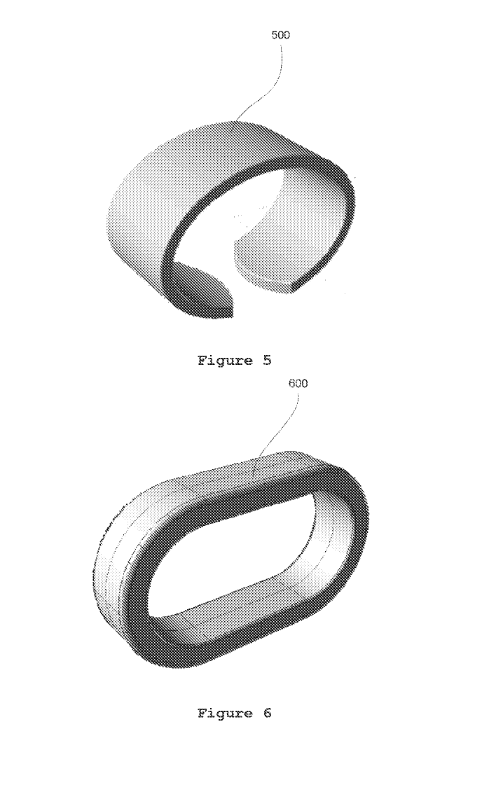

FIG. 5 illustrates a wristband or an armband alert unit 500, in accordance with one aspect. Alert unit 500 contains electronics and transducers and is worn by the user during sleep. Alert unit 500 is exemplary, and other alert units could be used. For example, the alert unit may be a wrist watch, a health and fitness wristband or armband, a necklace, a nose clip, a standalone unit with dedicated function of generating alerting signal only without other functions mentioned above. The fastening mechanism can be, but not limited to, a rubber band, elastic material, a watchband, a metal chain or any other means to attach the device to human wrists, arms, legs, chest and/or any other part of body for comfort and ease usage.

In some embodiments, alert unit 500 (and other alert units described herein) may include a vibratory function as a stimulus for the user. Different stimuli may also be used.

FIG. 6 illustrates a wristband alert unit 600, in accordance with one aspect. Unit 600 is an alternative design to alert unit 500 and may house the same electronics components and circuit to achieve the same function and performance.

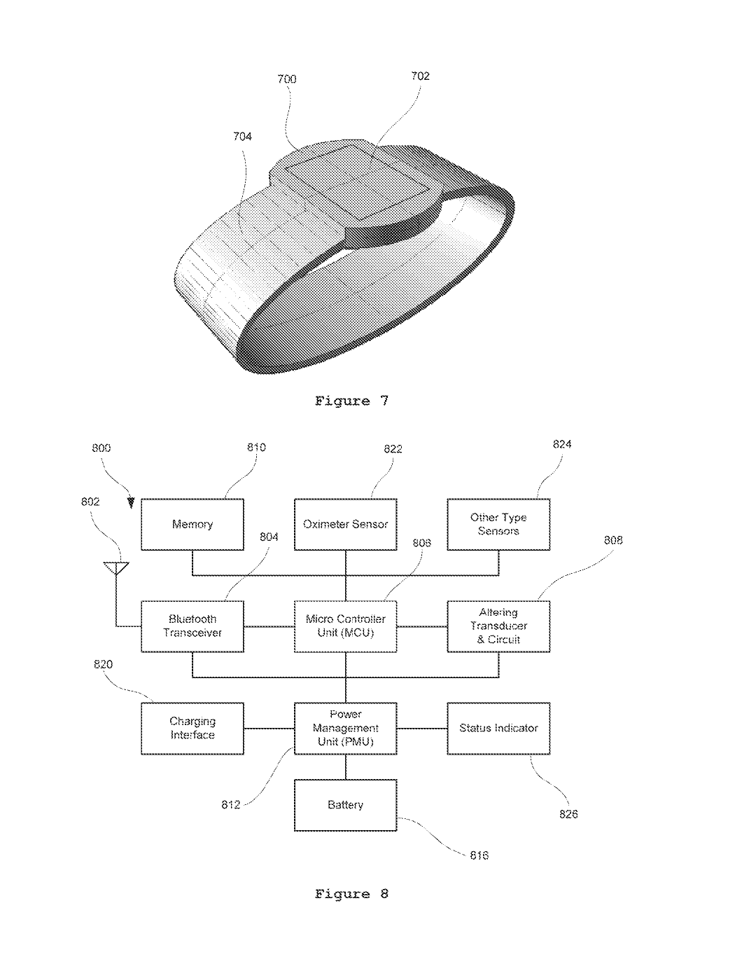

FIG. 7 illustrates an alert unit 700 integrated with a wrist-watch. The watch-type alert unit includes a display 702 and a fastening band 704 to attach the device to a user. Display 702 may be a touch sensitive panel to control the user interface and the alert unit 700. In some embodiments, alert unit may be a health fitness monitor such as heart rate monitor, a pedometer, oximeter (such as a pulse oximeter, for example), or a blood pressure monitor.

FIG. 8 illustrates a system block diagram 800 of an alert unit, in accordance with one aspect. Bluetooth antenna 802 may be used to establish wireless communication between the alerting unit and the smart mobile device via Bluetooth communication channels and protocol. Antenna 802 can be any of various antenna types, including chip antenna, PCB antenna, and 3D antenna printed onto the wristband mechanical structure. Connected to the Bluetooth antenna, the Bluetooth transceiver 804 transmits and receives the Bluetooth signal to and from the smart mobile device over the air. The Bluetooth transceiver 804 may comply with all Bluetooth and/or Bluetooth Low Energy standard protocols and profiles in order to work with all mobile devices that also support Bluetooth standards. Upon receiving alerting commands from a smart mobile device, the micro controller unit 806 can send the alerting signal to the Alerting Transducer & Circuit 808 to alert the user. The micro controller unit 806 can be a standard off-the-shelf devices such as MSP430 integrated circuits from TI or any other ICs that perform similar functions. Memory 810 stores the software and firmware codes for the micro controller unit 806 and temporary data. Alerting Transducer 808 may use an electro-vibrator to produce vibration. Micro Controller Unit 806 provides the overall control of all circuits within the alerting unit. Power Management Unit 812 can be standard off-the-shelf devices or ICs. The Power Management Unit 812 manages the standard rechargeable battery 816 charging process and provides the proper amount of energy from the battery to the entire circuit to achieve the best power efficiency. A rechargeable battery 816 provides energy during the normal use and will be recharged when it is not used during the non-sleep time. Charge interface 820 allows the standard AC charger to charge the battery via a standard micro USB connector. An oximeter 822 can be also implemented in the alerting unit to detect user's oxygen level during sleep. In case the oxygen level is lower than a predefined threshold due to snore or apnea, the data can be sent to the smart mobile device for further analysis. In some embodiments, the oximeter is a pulse oximeter. Once identified as a concern by the application software, the alerting commands can also be delivered from the smart mobile device to the alert unit to alert the user. Other type of sensors 824 can also be implemented for other type of detection and monitoring including, but not limited to, body temperature, heart rate, body movement etc. The collected data can be transmitted to the smart mobile device and recorded in the memory or a cloud server for future fitness and health analysis and improvement. The status indicator 826 can be a LED to indicate if the device is turned on before use and if the battery is low by lighting up with different colors and blinking pattern. The light strength of LED should be low to avoid disturbance of sleep and also save battery energy. The other types of indicators including audible device can also be implemented for other type of indications.

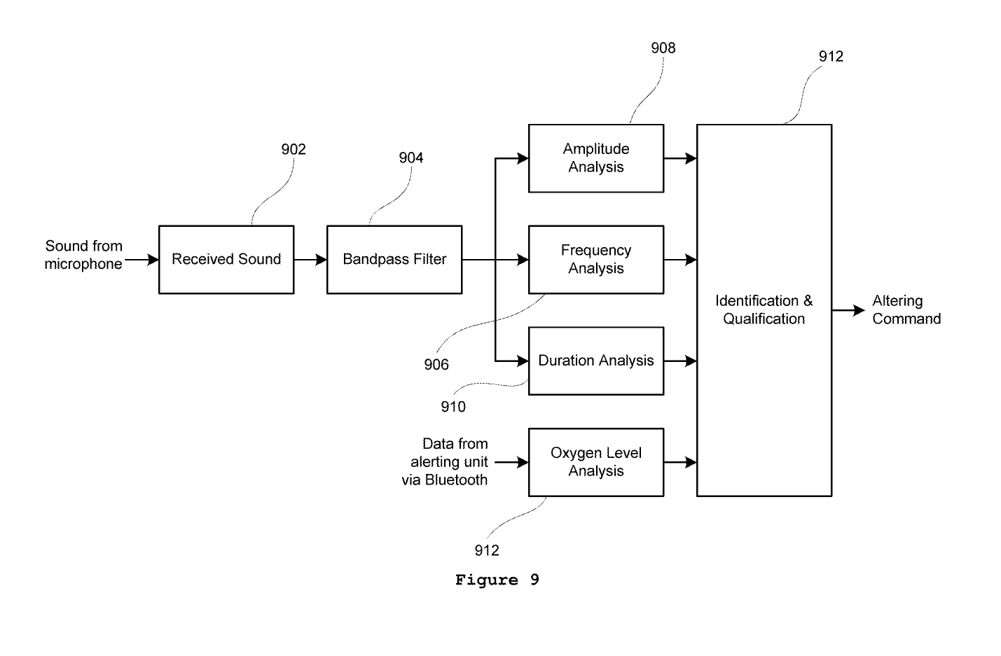

FIG. 9 illustrates a software architecture for detection, analyzing and identifying snore sound, in accordance with one aspect. This overall software architecture illustrates an embodiment for detecting and identifying the snoring sound received from the smart mobile device microphone. Received sound 902 is the data recorded in the memory from the microphone and converted to digital format. The data will be filtered by the band pass filter 904 to remove all unwanted sound signals that are not part of snoring signal. Once the sound signals are ready to be analyzed, the data will be submitted to frequency analysis module 906, amplitude analysis module 908 and duration analysis module 910. (In some embodiments, only one or a combination of two of these modules is used.) Each module will perform the analysis to identify if the signal meets the criteria of each category. If the signal meets all three analysis module's target requirements, the qualification and identification module 912 will issue an alerting command and the smart mobile device will send the command to the alerting unit via Bluetooth protocol. The algorithm with the application software may continue throughout the entire sleep duration until the user switches off the application software or the alerting module.

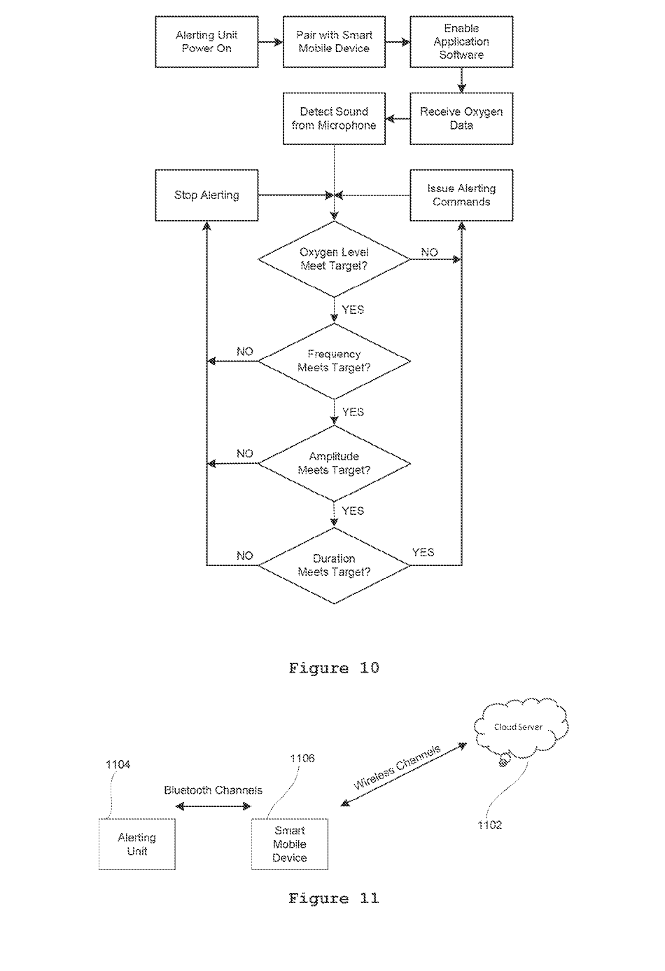

FIG. 10 illustrates a software flowchart of architecture implemented in the smart mobile device, in accordance with one aspect. The algorithm may be implemented in the application software in the smart mobile device.

In some embodiments, communication between the alert unit and the smart mobile device may be achieved through standard Bluetooth protocol and profiles. Once the alert unit is powered on, it can send a pairing request to the smart mobile device that has application software. Once paired, the smart mobile device may activate the application software and start the snoring monitoring. The application software enables the hardware including the microphone, core processor, and stores the data into the phone memory.

The application software may analyze the recorded signal's frequency, amplitude, duration and other patterns to determine if user is snoring. Upon identification, the application software may send an alerting signal via the smart mobile device's Bluetooth channels and protocols to the altering unit. Monitoring may be continuous and the alerting signal may end when the snoring signal is no longer detected. The application software will also record the signal throughout the entire sleep and the tracking of historical data that can be displayed to the user to indicate any improvement or how snoring pattern has been changed. The snoring data can also be uploaded to a cloud based server and share the data with a health or a medical professional for further analysis, as discussed later. In some embodiments, a smart mobile phone determines whether an audio signal includes characteristics of snoring by determining that at least one of a frequency, an amplitude, and a duration of the first audio signal meets a predetermined threshold.

FIG. 11 illustrates a cloud server 1102 configuration of connecting to a smart mobile device to store and share data for future analysis, in accordance with one aspect. Data from an alerting unit 1104 and/or a smart mobile device 1106 can be stored here. With permission from the user, the data can be shared with health and medical professionals for analysis.

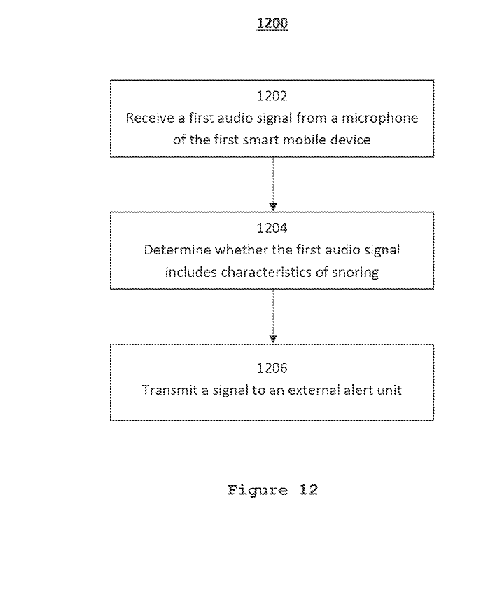

FIG. 12 illustrates a method 1200 of snore detection and correction, in accordance with one aspect. At block 1202, a first smart mobile device receives a first audio signal from a microphone of the first smart mobile device. At block 1204, the device determines that the first audio signal includes characteristics of snoring. In response to a determination that the first audio signal includes characteristics of snoring, the device at block 1206 transmits a signal to an external alert unit.

In some embodiments, determining that the first audio signal includes characteristics of snoring includes: receiving a communication from a second smart mobile device, wherein the communication comprises information about a second audio signal captured by the second smart mobile device; and determining whether at least one of the first audio signal and second audio signal is associated with a user of the first smart mobile device; in response to a determination that at least one of the first audio signal and second audio signal is not associated with a user of the first smart mobile device: foregoing transmission of the signal to the external alert unit; in response to a determination that at least one of the first audio signal and second audio signal is associated with a user of the first smart mobile device: transmitting the signal to the external alert unit. In a further aspect, determining whether at least one of the first audio signal and the second audio signal is associated with a user of the first smart mobile device comprises determining, from the information in the communication, at least one of a time of capture of the second audio signal at the second smart mobile device and an amplitude of the second audio signal.

In some embodiments, the alert unit is a wearable unit. In some embodiments, the alert unit is a vibratory unit.

In some embodiments, method 1200 includes receiving a measurement of an oxygen level of a user and wherein determining that the first audio signal includes characteristics of snoring further comprises determining whether the oxygen level is lower than a predetermined threshold. In further embodiments, receiving a measurement of an oxygen level comprises receiving the measurement from the alert unit.

In another embodiment, the first audio signal is recorded and the recorded audio signal is transmitted to an external server.

In another embodiment, method 1200 includes: receiving a third audio signal from the microphone of the first smart mobile device; determining whether the third audio signal includes characteristics of snoring; and in response to a determination that the third audio signal includes characteristics of snoring: transmitting a second signal to an alert unit. In a further embodiment, the second signal includes an instruction to increase an intensity of a stimulus produced by the alert unit. In a further embodiment, method 1200 includes determining whether a first snore associated with the first audio signal and a second snore associated with the third audio signal occurred within a predetermined period of time, and wherein the second signal includes an instruction to increase an intensity of a stimulus produced by the alert unit.

In some embodiments, determining that the first audio signal includes characteristics of snoring comprises determining that at least one of a frequency, an amplitude, and a duration of the first audio signal meets a predetermined threshold.

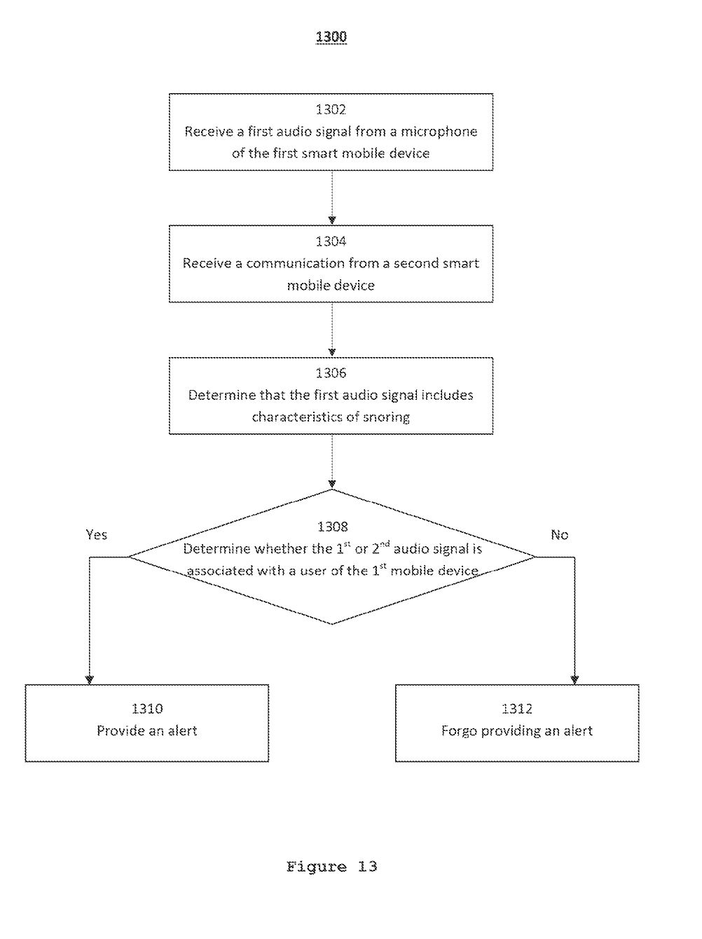

FIG. 13 illustrates a method 1300 of snore detection and correction, in accordance with one aspect. At block 1302, a first smart mobile device receives a first audio signal from a microphone of the first smart mobile device. At block 1304, the device receives a communication from a second smart mobile device, wherein the communication comprises information about a second audio signal captured by the second smart mobile device. At block 1306, the device determines that the first audio signal includes characteristics of snoring. In response to a determination that the first audio signal includes characteristics of snoring, the device at block 1308 determines whether at least one of the first audio signal and second audio signal is associated with a user of the first smart mobile device. In response to a determination that at least one of the first audio signal and second audio signal is associated with a user of the first smart mobile device, the device at block 1310 provides an alert. In response to a determination that at least one of the first audio signal and second audio signal is associated with a user of the first smart mobile device, the device at block 1312 forgoes providing an alert.

For ease of explanation, method 1300 includes block 1308 occurring in response to block 1306. As will be appreciated by one of skill in the art, the determinations in blocks 1306 and 1308 could be exchanged such that the decision diamond leading to blocks 1310 and 1312 result from the determination of whether the first audio signal includes characteristics of snoring, after it has been determination that at least one of the first audio signal and second audio signal is associated with a user of the first smart mobile device. In some embodiments, blocks 1306 and 1308 can be combined such in response to a determination that (a) at least one of the first audio signal and second audio signal is associated with a user of the first smart mobile device and (b) the first audio signal includes characteristics of snoring: provide an alert; and in response to a determination that (a) at least one of the first audio signal and second audio signal is associated with a user of the first smart mobile device or (b) the first audio signal does not include characteristics of snoring: forego providing an alert.

In some embodiments, providing an alert comprises transmitting a signal to an external alert unit.

In some embodiments, determining whether the second audio signal is associated with a user of the first smart mobile device comprises determining, from the information in the communication, at least one of a time of capture of the second audio signal at the second smart mobile device and an amplitude of a second audio signal.

The disclosure herein has primarily focused on snoring. However, it should be understood that the disclosure can be readily expanded to detect and monitor other sleep quality related parameters including oxygen level, heart rate, body temperature, body movement, coughing, sneezing, sleep walking, crying, sleep talking, asthma wheezing etc.

In some variations, the software applications may be executed via non-transitory computer storable medium having stored therein instructions, which when executed by a device, cause the device to perform a method of detecting and correcting snoring, such as methods disclosed herein including, for example, methods 1200 and 1300. In other variations, an electronic device may have a processor and a memory coupled to the processor to store instructions, which when executed by the processor, cause the processor to perform operations to generate an application programming interface (API) that allows an API-calling component to display a plurality of informational displays on an user interface and/or detecting a snore and/or sending a signal.

The embodiments described above may operate on one or more server computers that allow interconnected computer network users to participate in a system for detecting and correcting snoring. This can be accomplished, for example, by users accessing non-transitory computer readable media on a server computer via the internet. This readable media contains the program instructions for accomplishing various steps described above. In the context of this document, a computer-readable storage medium can be any medium that can contain or store programming for use by or in connection with an instruction execution system, apparatus, or device. Such computer readable media may be stored on a memory, where a memory is any device capable of storing a computer readable medium and capable of being accessed by a computer. A memory may include additional features. A computer may include a processor. A processor can be any device suitable to access a memory and execute a program stored thereon.

Although the present invention has been fully described in connection with embodiments thereof with reference to the accompanying drawings, it is to be noted that various changes and modifications will become apparent to those skilled in the art. Such changes and modifications are to be understood as being included within the scope of the present invention. The various embodiments of the invention should be understood that they have been presented by way of example only, and not by way of limitation. Although the invention is described above in terms of various exemplary embodiments and implementations, it should be understood that the various features and functionality described in one or more of the individual embodiments are not limited in their applicability to the particular embodiment with which they are described. They instead can be applied, alone or in some combination, to one or more of the other embodiments of the invention, whether or not such embodiments are described, and whether or not such features are presented as being a part of a described embodiment. Thus the breadth and scope of the invention should not be limited by any of the above-described exemplary embodiments.

It will be appreciated that, for clarity purposes, the above description has described embodiments of the invention with reference to different functional units and processors. However, it will be apparent that any suitable distribution of functionality between different functional units, processing logic elements or domains may be used without detracting from the invention. For example, functionality illustrated to be performed by separate processing logic elements, or controllers, may be performed by the same processing logic element, or controller. Hence, references to specific functional units are only to be seen as references to suitable means for providing the described functionality, rather than indicative of a strict logical or physical structure or organization.

As will be readily understood by one of ordinary skill in the art, the interfaces described herein could be utilized on a variety of devices, such as personal computers, laptops, tablets, and smartphones, for example.

As used herein, “a” or “an” means “at least one” or “one or more.” It is understood that aspects and embodiments of the invention described herein include “consisting” and/or “consisting essentially of” aspects and embodiments.

Other objects, advantages and features of the present invention will become apparent from the following specification taken in conjunction with the accompanying drawings.

1. A method of detecting snoring comprising:
at a first smart mobile device:
receiving a first audio signal from a microphone of the first smart mobile device;
determining that the first audio signal includes characteristics of snoring; and
in response to a determination that the first audio signal includes characteristics of snoring:
transmitting a signal to an external alert unit.

2. The method of claim 1, wherein determining that the first audio signal includes characteristics of snoring further comprises:
receiving a communication from a second smart mobile device, wherein the communication comprises information about a second audio signal captured by the second smart mobile device;
determining whether at least one of the first audio signal and second audio signal is associated with a user of the first smart mobile device;
in response to a determination that at least one of the first audio signal and second audio signal is associated with a user of the first smart mobile device:
transmitting the signal to the external alert unit; and
in response to a determination that at least one of the first audio signal and second audio signal is not associated with a user of the first smart mobile device:
foregoing transmission of the signal to the external alert unit.

3. The method of claim 2, wherein determining whether at least one of the first audio signal and second audio signal is associated with a user of the first smart mobile device comprises determining, from the information in the communication, at least one of a time of capture of the second audio signal at the second smart mobile device and an amplitude of the second audio signal.

4. The method of claim 1, wherein the alert unit is a wearable unit.

5. The method of claim 1, wherein the alert unit is a vibratory unit.

6. The method of claim 1, further comprising receiving a measurement of an oxygen level of a user and wherein determining that the first audio signal includes characteristics of snoring further comprises determining whether the oxygen level is lower than a predetermined threshold.

7. The method of claim 6, wherein receiving a measurement of an oxygen level comprises receiving the measurement from the alert unit.

8. The method of claim 1, wherein the first audio signal is recorded and the recorded audio signal is transmitted to an external server.

9. The method of claim 1, further comprising:
receiving a third audio signal from the microphone of the first smart mobile device;
determining whether the third audio signal includes characteristics of snoring; and
in response to a determination that the third audio signal includes characteristics of snoring:
transmitting a second signal to an alert unit.

10. The method of claim 9, wherein the second signal includes an instruction to increase an intensity of a stimulus produced by the alert unit.

11. The method of claim 9, further comprising determining whether a first snore associated with the first audio signal and a second snore associated with the third audio signal occurred within a predetermined period of time, and wherein the second transmitted signal includes an instruction to increase an intensity of a stimulus produced by the alert unit.

12. The method of claim 1, wherein determining that the first audio signal includes characteristics of snoring comprises determining that at least one of a frequency, an amplitude, and a duration of the first audio signal meets a predetermined threshold.

13. A method of detecting snoring comprising:
at a first smart mobile device:
receiving a first audio signal from a microphone of the first smart mobile device;
receiving a communication from a second smart mobile device, wherein the communication comprises information about a second audio signal captured by the second smart mobile device;
determining whether the first audio signal includes characteristics of snoring; and
determining whether at least one of the first audio signal and second audio signal is associated with a user of the first smart mobile device;
in response to a determination that (a) at least one of the first audio signal and second audio signal is associated with a user of the first smart mobile device and (b) the first audio signal includes characteristics of snoring:
providing an alert; and
in response to a determination that (a) at least one of the first audio signal and second audio signal is not associated with a user of the first smart mobile device or (b) the first audio signal does not include characteristics of snoring:
forgoing providing an alert.

14. The method of claim 13, wherein providing an alert comprises transmitting a signal to an external alert unit.

15. The method of claim 13, wherein determining whether at least one of the first audio signal and second audio signal is associated with a user of the first smart mobile device comprises determining, from the information in the communication, at least one of a time of capture of the second audio signal at the second smart mobile device and an amplitude of a second audio signal.

16. A non-transitory computer-readable storage medium storing one or more programs, the one or more programs comprising instructions, which when executed by a first smart mobile device, cause the device to:
determine whether a first audio signal, received from a microphone of the first smart mobile device, includes characteristics of snoring; and
in response to a determination that the first audio signal includes characteristics of snoring:
transmit a signal to an external alert unit.

17. A non-transitory computer-readable storage medium storing one or more programs, the one or more programs comprising instructions, which when executed by a first smart mobile device, cause the device to:
determine whether a first audio signal, received from a microphone of the first smart mobile device, includes characteristics of snoring; and
determine whether at least one of the first audio signal and second audio signal is associated with a user of the first smart mobile device, wherein the second audio signal is detected by a second smart mobile device;
in response to a determination that (a) at least one of the first audio signal and second audio signal is associated with a user of the first smart mobile device and (b) the first audio signal includes characteristics of snoring:
provide an alert; and
in response to a determination that (a) at least one of the first audio signal and second audio signal is not associated with a user of the first smart mobile device or (b) the first audio signal does not include characteristics of snoring:
forgo providing an alert.
